(19) United States
(12) Patent Application Publication
(10) Pub. No.: US 2019/0156958 A1
(43) Pub. Date: May 23, 2019

(54) HEALTHCARE NETWORK

(71) Applicant: Siemens Healthcare GmbH, Erlangen (DE)

(72) Inventors: Ulrich HARTUNG, Langensendelbach (DE); Christian SPITZNER, Forchheim (DE); Iwona MARKUSZEWSKA, Forchheim (DE)

(73) Assignee: Siemens Healthcare GmbH, Erlangen (DE)

(21) Appl. No.: 16/196,377

(22) Filed: Nov. 20, 2018

(30) Foreign Application Priority Data
Nov. 23, 2017 (EP) 17203294.8

(51) Int. Cl. G16H 50/70; G16H 10/60

(52) U.S. Cl. CPC G16H 50/70; G16H 10/60

(57) ABSTRACT

A system operable to provide healthcare data to a user device in a healthcare network includes a plurality of data sources including healthcare data. The system maintains a first plurality of associations between a first plurality of the data sources and healthcare data stored therein. In response to a request for healthcare data from the user device, the system determines whether the requested healthcare data can be retrieved based on the first plurality of associations. If the requested healthcare data cannot be retrieved, the system causes a second association in relation to a second data source including the requested healthcare data to be detected and stored. The system retrieves the requested healthcare data from the second data source and transmits it for receipt by the user device.

### PRIORITY STATEMENT

The present application hereby claims priority under 35 U.S.C. § 119 to European patent application number EP 17203294.8 filed Nov. 23, 2017, the entire contents of which are hereby incorporated herein by reference.

### FIELD

Embodiments of the invention described herein generally relate, but not exclusively, to systems, methods and computer programs operable to provide healthcare data to a user device in a healthcare network.

### BACKGROUND

A healthcare network enables accumulation and/or provisioning of healthcare, clinical and/or medical data. The healthcare network comprises a plurality of systems including devices and software applications. The devices may include medical devices (e.g. an ultrasound system), user devices for use by medical professionals and/or patients, servers and medical data sources. Software applications operate on, or in conjunction with or are accessible via, the devices, and may include communication applications that facilitate communication between devices on the healthcare network, medical applications configured to process healthcare data, applications configured to manage patient information, applications for managing knowledge in a healthcare environment, and applications configured to manage medical records.

Typically, a healthcare network comprises several disparate proprietary, off-the-shelf, structured and/or unstructured data sources, which are rarely integrated so as to operate homogeneously. A healthcare professional, when analysing healthcare data associated with a patient, may need healthcare data from different data sources at different stages of the analysis. In addition, healthcare professionals from different disciplines may need different healthcare data for their analysis. For example, different members of a tumour board, which is a multidisciplinary group of healthcare professionals that reviews ongoing cancer cases, may need access to different healthcare data. However, healthcare professionals have to manually retrieve healthcare data from various data sources to get all the relevant information required.

### SUMMARY

In a first embodiment, there is provided a system operable to provide healthcare data to a user device in a healthcare network, the healthcare network comprising a plurality of data sources comprising healthcare data, the system comprising at least one processor and at least one memory including computer program code, the at least one memory and the computer program code configured to, with the at least one processor, cause the system at least to perform the steps of:

maintaining, in a database, a first plurality of associations between a first plurality of the data sources and healthcare data stored therein;

receiving, from the user device, a request for healthcare data, wherein the requested healthcare data is stored in a second of the data sources, the second data source being different to the first plurality of data sources;

determining whether the requested healthcare data can be retrieved based on the first plurality of associations; selectively, based on an outcome of the determining, causing a second association between the second data source and the requested healthcare data to be detected and stored in the database;

retrieving the requested healthcare data from the second data source; and

transmitting the retrieved healthcare data for receipt by the user device.

In a second embodiment, there is provided a method of providing healthcare data to a user device in a healthcare network, the healthcare network comprising a plurality of data sources comprising healthcare data, the method comprising:

maintaining, in a database, a first plurality of associations between a first plurality of the data sources and healthcare data stored therein;

receiving, from the user device, a request for healthcare data, wherein the requested healthcare data is stored in a second of the data sources, the second data source being different to the first plurality of data sources;

determining whether the requested healthcare data can be retrieved based on the first plurality of associations; selectively, based on an outcome of the determining, causing a second association between the second data source and the requested healthcare data to be detected and stored in the database;

retrieving the requested healthcare data from the second data source; and

transmitting the retrieved healthcare data for receipt by the user device.

In a third embodiment, there is provided a computer program comprising a set of instructions, which, when executed by a computerised device, cause the computerised device to perform a method of providing healthcare data to a user device in a healthcare network, the healthcare network comprising a plurality of data sources comprising healthcare data, the method comprising, at the computerised device:

maintaining, in a database, a first plurality of associations between a first plurality of the data sources and healthcare data stored therein;

receiving, from the user device, a request for healthcare data, wherein the requested healthcare data is stored in a second of the data sources, the second data source being different to the first plurality of data sources;

determining whether the requested healthcare data can be retrieved based on the first plurality of associations; selectively, based on an outcome of the determining, causing a second association between the second data source and the requested healthcare data to be detected and stored in the database;

retrieving the requested healthcare data from the second data source; and

transmitting the retrieved healthcare data for receipt by the user device.

In a fourth embodiment, there is provided a system operable to provide healthcare data to a user device in a healthcare network, the healthcare network comprising a plurality of data sources comprising healthcare data, the system comprising at least one processor and at least one memory including computer program code, the at least one memory and the computer program code configured to, with the at least one processor, cause the system at least to perform the steps of:

maintaining, in a database, a plurality of patient models, each patient model comprising a virtual map associating healthcare data related to at least one patient and a first plurality of the data sources;

receiving, from the user device, a request for healthcare data;

identifying at least one patient model in dependence on the requested healthcare data;

retrieving the requested healthcare data using the identified at least one patient model; and

transmitting the retrieved healthcare data for receipt by the user device.

In a fifth embodiment, there is provided a method of providing healthcare data to a user device in a healthcare network, the healthcare network comprising a plurality of data sources comprising healthcare data, the method comprising:

maintaining, in a database, a plurality of patient models, each patient model comprising a virtual map associating healthcare data related to at least one patient and a first plurality of the data sources;

receiving, from the user device, a request for healthcare data;

identifying at least one patient model in dependence on the requested healthcare data;

retrieving the requested healthcare data using the identified at least one patient model; and

transmitting the retrieved healthcare data for receipt by the user device.

In a sixth embodiment, there is provided a computer program comprising a set of instructions, which, when executed by a computerised device, cause the computerised device to perform a method of providing healthcare data to a user device in a healthcare network, the healthcare network comprising a plurality of data sources comprising healthcare data, the method comprising, at the computerised device:

maintaining, in a database, a plurality of patient models, each patient model comprising a virtual map associating healthcare data related to at least one patient and a first plurality of the data sources;

receiving, from the user device, a request for healthcare data;

identifying at least one patient model in dependence on the requested healthcare data;

retrieving the requested healthcare data using the identified at least one patient model; and

transmitting the retrieved healthcare data for receipt by the user device.

In another embodiment, there is provided a system operable to provide healthcare data to a user device in a healthcare network, the healthcare network including a plurality of data sources including healthcare data, the system comprising:

at least one processor; and

at least one memory storing computer program code, the at least one memory and the computer program code being configured to, with the at least one processor, cause the system to at least:
maintain, in a database, a first plurality of associations between a first plurality of the data sources and healthcare data stored in the database;
receive, from the user device, a request for healthcare data, wherein the healthcare data requested is stored in a second data source, the second data source being different from the first plurality of data sources;
determine whether the healthcare data requested is retrievable based on the first plurality of associations;
selectively, based on a result determined, causing a second association between the second data source and the healthcare data requested to be detected and stored in the database;
retrieve the healthcare data requested from the second data source; and
transmit the healthcare data retrieved for receipt by the user device.

In another embodiment, there is provided a method of providing healthcare data to a user device in a healthcare network, the healthcare network including a plurality of data sources including healthcare data, the method comprising:

maintaining, in a database, a first plurality of associations between a first plurality of the data sources and healthcare data stored in the database;

receiving, from the user device, a request for healthcare data, wherein the healthcare data requested is stored in a second data source, different from the first plurality of data sources;

determining whether the healthcare data requested is retrievable based on the first plurality of associations;

selectively, based on a result of the determining, causing a second association between the second data source and the healthcare data requested to be detected and stored in the database;

retrieving the healthcare data requested from the second data source; and

transmitting the healthcare data retrieved for receipt by the user device.

In another embodiment, there is provided a non-transitory computer readable medium storing a computer program, including a set of instructions which, when executed by a computerized device, cause the computerized device to perform a method of providing healthcare data to a user device in a healthcare network, the healthcare network comprising a plurality of data sources comprising healthcare data, the method comprising:

maintaining, in a database, a first plurality of associations between a first plurality of the data sources and healthcare data stored in the database;

receiving, from the user device, a request for healthcare data, wherein the healthcare data requested is stored in a second data source, different from the first plurality of data sources;

determining whether the healthcare data requested is retrievable based on the first plurality of associations;

selectively, based on a result of the determining, causing a second association between the second data source and the healthcare data requested to be detected and stored in the database;

retrieving the healthcare data requested from the second data source; and

transmitting the healthcare data retrieved for receipt by the user device.

### BRIEF DESCRIPTION OF THE DRAWINGS

FIG. 1 shows a schematic block diagram of an example of a healthcare network in accordance with examples;

FIG. 2 shows a flow diagram depicting an example of processing data in accordance with examples;

FIG. 3 shows a schematic block diagram of an example of a healthcare data provisioning framework in accordance with examples;

FIG. 4 shows a schematic block diagram of an example of a healthcare data provisioning framework in accordance with examples;

FIG. 5 shows an example patient model in accordance with examples;

FIG. 6 shows a flow diagram depicting an example of processing data in accordance with examples;

FIG. 7 shows a schematic block diagram of an example of an interface on a user device in accordance with examples; and

FIGS. 8-12 present examples of the interface shown with reference to FIG. 7.

### DETAILED DESCRIPTION OF EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. The present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “exemplary” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuity such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without subdividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Bluray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Most of the aforementioned components, in particular the identification unit, can be implemented in full or in part in the form of software modules in a processor of a suitable control device or of a processing system. An implementation largely in software has the advantage that even control devices and/or processing systems already in use can be easily upgraded by a software update in order to work in the manner according to at least one embodiment of the invention.

In a first embodiment, there is provided a system operable to provide healthcare data to a user device in a healthcare network, the healthcare network comprising a plurality of data sources comprising healthcare data, the system comprising at least one processor and at least one memory including computer program code, the at least one memory and the computer program code configured to, with the at least one processor, cause the system at least to perform the steps of:

maintaining, in a database, a first plurality of associations between a first plurality of the data sources and healthcare data stored therein;

receiving, from the user device, a request for healthcare data, wherein the requested healthcare data is stored in a second of the data sources, the second data source being different to the first plurality of data sources;

determining whether the requested healthcare data can be retrieved based on the first plurality of associations; selectively, based on an outcome of the determining, causing a second association between the second data source and the requested healthcare data to be detected and stored in the database;

retrieving the requested healthcare data from the second data source; and

transmitting the retrieved healthcare data for receipt by the user device.

Therefore, the system advantageously integrates disparate data sources by maintaining associations between various data sources and healthcare data stored therein. If the requested healthcare data cannot be retrieved based on the maintained associations (i.e. the first plurality of associations), then the system may cause the second association to be detected, thereby selectively causing unknown data source(s) to be identified and associations in respect thereof to be stored. Subsequent requests for healthcare data from the second data source may be processed by the system using the second association, which is now known thereto, by retrieving and transmitting the requested healthcare data for receipt by the user device.

In the event that an association corresponding to requested healthcare data can be identified on the basis of the first plurality of associations, the system retrieves and transmits the requested healthcare data for receipt by the user device. Therefore, the system causes the second association to be selectively identified in dependence on whether the requested healthcare data can be retrieved based on the first plurality of associations, thereby increasing average response times for responding to requests for healthcare data.

The system may use an application detect which of the data sources comprises the requested healthcare data, thereby detecting the second association. The application may have crawling functionality. Therefore, embodiments enable a healthcare data provisioning framework in which the system operates alongside an application configured to detect associations that are unknown to the system.

The system may additionally remove an association from the first plurality of associations, thereby ceasing access to the corresponding healthcare data via the healthcare network. The system may, in response to receiving data indicative of a change between the second data source and healthcare data stored therein, update the second association, thereby continuing to facilitate access to the healthcare data stored in the second data source via the healthcare network. Therefore, embodiments facilitate access to up-to-date healthcare data.

The system may create one or more virtual maps based on the known associations, thereby creating one or more patient models. The or each patient model links related healthcare data. The system may use the patient model(s) to acquire relevant healthcare data in response to a request therefor.

The system may maintain a state parameter indicative of a current stage of analysis, and use it to cause at least part of the retrieved healthcare data to be displayed on a user terminal. The state parameter may, for example, be indicative of a clinical stage, such diagnosis, treatment etc.

The system may monitor prior actions of a user when analysing healthcare data associated with at least some of the different stages and may control display of at least a part of the acquired healthcare data on the user device based on a combination of the state parameter and the monitoring. Therefore, embodiments reduce cognitive burden by pre-processing acquired healthcare data based on a user's style of working.

The system may, in response to a user input, cause the user device to acquire further healthcare data based on a combination of the input and the state parameter and using the patient model, and may cause the user device to present at least a part of the acquired further healthcare data. Therefore, embodiments are responsive to inputs from users at various stages, and use the inputs to automatically acquire and display further healthcare data. The system may, additionally or alternatively, process the retrieved healthcare data based on a predetermined parameter, and thereafter cause at least a part of the processed healthcare data to be presented. The predetermined parameter may, for example, comprise data indicative of a relevant frame of reference, such as a patient, for use in pre-processing of healthcare data prior to presentation on a user device.

The system may, additionally or alternatively, maintain a context parameter indicative of a clinical step associated with analysis of the received data and is a step in a clinical stage or process. For example, the context parameter may be indicative of a step of premedication in a clinical process of bronchoscopy.

The system may control display of at least a part of the filtered healthcare data on the user device based on a combination of the state parameter and the context parameter, thereby providing relevant healthcare date for a current step of assessment.

In a second embodiment, there is provided a method of providing healthcare data to a user device in a healthcare network, the healthcare network comprising a plurality of data sources comprising healthcare data, the method comprising:

maintaining, in a database, a first plurality of associations between a first plurality of the data sources and healthcare data stored therein;

receiving, from the user device, a request for healthcare data, wherein the requested healthcare data is stored in a second of the data sources, the second data source being different to the first plurality of data sources;

determining whether the requested healthcare data can be retrieved based on the first plurality of associations; selectively, based on an outcome of the determining, causing a second association between the second data source and the requested healthcare data to be detected and stored in the database;

retrieving the requested healthcare data from the second data source; and

transmitting the retrieved healthcare data for receipt by the user device.

In a third embodiment, there is provided a computer program comprising a set of instructions, which, when executed by a computerised device, cause the computerised device to perform a method of providing healthcare data to a user device in a healthcare network, the healthcare network comprising a plurality of data sources comprising healthcare data, the method comprising, at the computerised device:

maintaining, in a database, a first plurality of associations between a first plurality of the data sources and healthcare data stored therein;

receiving, from the user device, a request for healthcare data, wherein the requested healthcare data is stored in a second of the data sources, the second data source being different to the first plurality of data sources;

determining whether the requested healthcare data can be retrieved based on the first plurality of associations; selectively, based on an outcome of the determining, causing a second association between the second data source and the requested healthcare data to be detected and stored in the database;

retrieving the requested healthcare data from the second data source; and

transmitting the retrieved healthcare data for receipt by the user device.

In a fourth embodiment, there is provided a system operable to provide healthcare data to a user device in a healthcare network, the healthcare network comprising a plurality of data sources comprising healthcare data, the system comprising at least one processor and at least one memory including computer program code, the at least one memory and the computer program code configured to, with the at least one processor, cause the system at least to perform the steps of:

maintaining, in a database, a plurality of patient models, each patient model comprising a virtual map associating healthcare data related to at least one patient and a first plurality of the data sources;

receiving, from the user device, a request for healthcare data;

identifying at least one patient model in dependence on the requested healthcare data;

retrieving the requested healthcare data using the identified at least one patient model; and

transmitting the retrieved healthcare data for receipt by the user device.

Therefore, the system advantageously integrates disparate data sources by maintaining patient models comprising a virtual map associating healthcare data related to patient(s) and the first plurality of associations, and using the relevant patient model(s) for retrieving the requested healthcare data.

In the event that an association corresponding to requested healthcare data can be identified using the patient model(s), the system retrieves and transmits the requested healthcare data for receipt by the user device. If the requested healthcare data cannot be retrieved using the patient model(s), then the system causes a second association between a second data source and the requested healthcare data stored therein to be detected, wherein the requested healthcare data is stored in the second data source. Therefore, the system causes the second association to be selectively detected in dependence on whether the requested healthcare data can be retrieved based on the first plurality of associations, thereby increasing average response times for responding to requests for healthcare data.

The system may use an application to detect which of the data sources comprises the requested healthcare data, thereby detecting the second association. The application may have crawling functionality. Therefore, embodiments enable a healthcare data provisioning framework in which the system operates alongside an application configured to detect associations that are unknown to the system.

The system may additionally remove an association from the first plurality of associations, thereby ceasing access to the corresponding healthcare data via the healthcare network. In this case, the system may update the relevant of the patient model(s) subsequent to removal of an association.

The system may, in response to receiving data indicative of a change between the second data source and healthcare data stored therein, update the second association and the relevant patient model(s), thereby continuing to facilitate access to the healthcare data stored in the second data source via the healthcare network. Therefore, embodiments facilitate access to up-to-date healthcare data.

The system may maintain a state parameter indicative of a current stage of analysis, and use it to cause at least part of the retrieved healthcare data to be displayed on a user terminal. The state parameter may, for example, be indicative of a clinical stage, such diagnosis, treatment etc.

The system may monitor prior actions of a user when analysing healthcare data associated with at least some of the different stages and may control display of at least a part of the acquired healthcare data on the user device based on a combination of the state parameter and the monitoring. Therefore, embodiments reduce cognitive burden by pre-processing acquired healthcare data based on a user's style of working.

The system may, in response to a user input, cause the user device to acquire further healthcare data based on a combination of the input and the state parameter and using the relevant patient model(s), and may cause the user device to present at least a part of the acquired further healthcare data. Therefore, embodiments are responsive to inputs from users at various stages, and use the inputs to automatically acquire and display further healthcare data. The system may, additionally or alternatively, process the retrieved healthcare data based on a predetermined parameter, and thereafter cause at least a part of the processed healthcare data to be presented. The predetermined parameter may, for example, comprise data indicative of a relevant frame of reference, such as a patient, for use in pre-processing of healthcare data prior to presentation on a user device.

The system may, additionally or alternatively, maintain a context parameter indicative of a clinical step associated with analysis of the received data and is a step in a clinical stage or process. For example, the context parameter may be indicative of a step of premedication in a clinical process of bronchoscopy.

The system may control display of at least a part of the filtered healthcare data on the user device based on a combination of the state parameter and the context parameter, thereby providing relevant healthcare date for a current step of assessment.

In a fifth embodiment, there is provided a method of providing healthcare data to a user device in a healthcare network, the healthcare network comprising a plurality of data sources comprising healthcare data, the method comprising:

maintaining, in a database, a plurality of patient models, each patient model comprising a virtual map associating healthcare data related to at least one patient and a first plurality of the data sources;

receiving, from the user device, a request for healthcare data;

identifying at least one patient model in dependence on the requested healthcare data;

retrieving the requested healthcare data using the identified at least one patient model; and

transmitting the retrieved healthcare data for receipt by the user device.

In a sixth embodiment, there is provided a computer program comprising a set of instructions, which, when executed by a computerised device, cause the computerised device to perform a method of providing healthcare data to a user device in a healthcare network, the healthcare network comprising a plurality of data sources comprising healthcare data, the method comprising, at the computerised device:

maintaining, in a database, a plurality of patient models, each patient model comprising a virtual map associating healthcare data related to at least one patient and a first plurality of the data sources;

receiving, from the user device, a request for healthcare data;

identifying at least one patient model in dependence on the requested healthcare data;

retrieving the requested healthcare data using the identified at least one patient model; and

transmitting the retrieved healthcare data for receipt by the user device.

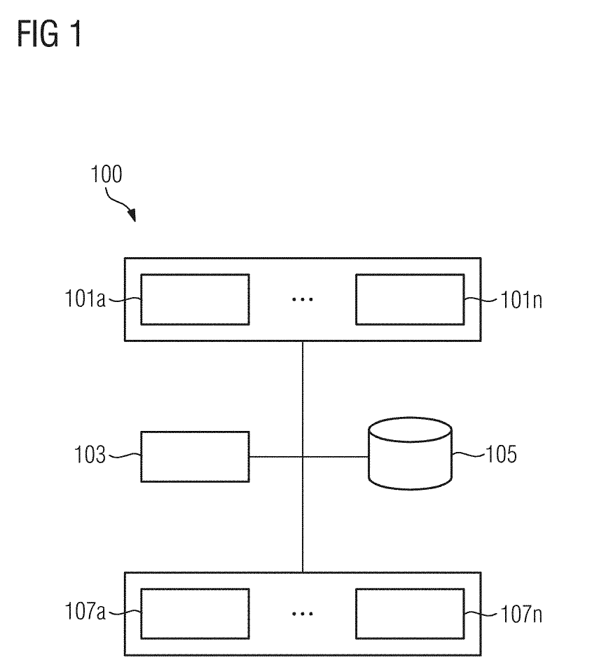

Referring to FIG. 1, there is shown schematically an example of a healthcare network 100. The healthcare network 100 comprises a number of systems. The healthcare network 100 may correspond to systems in one healthcare institution or multiple healthcare institutions. The term “system” is used herein to denote an entity (or entities) in the healthcare network 100. A system may be embodied in the form of apparatus, hardware, software, a function, a virtualized resource, etc., or any combination of same. A healthcare network can comprise at least some different and/or additional components to those shown in FIG. 1.

The healthcare network 100 comprises one or more user devices 101a-n configured for use by a healthcare professional or a patient. The user devices 101a-n may comprise a smart watch, a smart telephone, a tablet computer and/or a personal computer. The user devices 101a-n may comprise an application for presenting healthcare data. At least some of the user devices 101a-n may be communicatively coupled to each other via, for example, a local area network, metropolitan area network or a wide area network, and may have access to systems external to the healthcare network 100 via a Candidate Access Network (CAN) (not shown). The user devices 101a-n may comprise one or more processors (not shown) for performing various data processing operations according to examples and/or one or more memories (not shown) for storing various data according to examples.

The CAN may, for example, be a wireless access network or a wired access network. Examples of wireless access networks include, but are not limited to, Wireless Local Area Networks (WLANs) and mobile radio access networks. An example of a mobile radio access network is an Evolved Universal Terrestrial Radio Access Network (E-UTRAN). An example of a wired access network is an Asymmetric Digital Subscriber Line (ADSL).

The healthcare network 100 may additionally comprise or facilitate access to one or more servers (not shown) configured to provision applications and data to the user devices 101a-n and other systems, a plurality of data sources 107a-n configured to provision healthcare data, a statistical decision support system, a clinical decision support system, a population health management system and/or one or more medical devices (for example, an ultrasound system).

The data sources 107a-n may be structured or unstructured, and may comprise a mix of disparate proprietary and off-the-shelf data sources that are located internally or externally to the healthcare network 100. The data sources 107a-n may, for example, include an Electronic Medical Records (EMR), a Picture Archiving Communication Systems (PACS), an Emergency Care Summary (ECS), a Key Information Summary (KIS), an Electronic Healthcare Record (EHR), an Electronic Patient Record (EPR) data source; and/or a Personal Health Record (PHR) data source. At least some of the data sources 107a-n may be configured to communicate via a corresponding communications interface providing a communications channel and/or an application programming interface for enabling communication therewith. The health industry standards do not mandate all operational embodiments required for fully integrating data sources used in healthcare networks. In addition, there is very little guidance in the standards regarding integration of data sources with other systems in a healthcare network, specifically ones incorporating new technologies and/or involving multiple vendors. For example, some of the data sources 107a-n may conform to standards relevant for the medical industry, such as Health Level Seven (HL7) Fast Healthcare Interoperability Resources (FHIR) standard and/or Digital Imaging and Communications in Medicine (DICOM) standard, and/or conform to standards relevant for the digital communications industry, such as Representational State Transfer (REST) and/or Internet Protocol (IP).

At least some of the user devices 101a-n have access to a system 103 operable to provide healthcare data (also referred to as healthcare information). The healthcare data comprises clinical data (also referred to as clinical information) and/or medical data (also referred to as medical information). Clinical data comprises data collected during the course of ongoing patient care and/or as part of a formal clinical trial program, such as drug testing program. Clinical data may comprise: electronic health records of one or more patients, patient/disease registries indicative of data relating to chronic conditions such as Alzheimer's, clinical trials data, data for use in, for example, clinical or statistical decision support systems and/or anonymised patient medical records for, for example, use by a population health management system. Medical data comprises administrative, regulatory and R&D (research and development) data, guidelines and/or legal regulations.

The system 103 may be implemented on a single computer or a network of computers. In examples, the system 103 may be a central or location-specific distributed system. The system 103 may be located internally or externally to the healthcare network 100, and be operable to provide healthcare data to user devices or systems in one or a plurality of healthcare networks. The system 103 comprises one or more processors (not shown) for performing various data processing operations according to examples. The system 103 comprises or otherwise has access to a database 105 for storing various data according to examples. The database 105 may comprise one or more memories (not shown). The memory may be volatile so that data stored therein may need to be re-learnt upon failure/re-boot.

The database 105 maintains a first plurality of associations (also referred to as known associations) between a first plurality of the data sources 107a-n (also referred to as known data sources) and healthcare data stored therein. The mapping between the known data sources 107a-n and at least some of the healthcare data stored therein is recognised by the system 103, and can therefore be used for providing healthcare data to one or more of the user devices 101a-n and/or other systems, such as clinical or statistical decision support systems. The first plurality of associations may, for example, be known to the system 103 on the basis of one or more configuration parameters manually or automatically provided to the system 103 as part of the initial setup or otherwise. The system 103 may, additionally or alternatively, learn about associations between data sources and healthcare data stored therein using an application as will be described further below.

The known associations may be used by the system 103 to generate individual, collective, statistical, clinical or population patient models for use by healthcare professionals. The or each patient model is a virtual map of data sources recognised by the system 103 on the basis of the known associations, and is indicative of healthcare data relevant thereto. For example, an individual patient model comprises a virtual map associating healthcare data related to a patient based on the known associations. Collective patient models may correspond to a group of linked patients, such as patients linked by a blood relationship, and in this case the patient model comprises a virtual map associating healthcare data related to the group of patients based on the known associations. Statistical patient models may correspond to a group of patients sharing a clinical relationship, such as medical condition, for use in statistical decision support systems. Clinical patient models may correspond to a group of patients sharing a clinical relationship, such as medical condition, for use in clinical decision support systems. Population patient models may correspond to a group of related or unrelated patients for use in population health management systems, which aggregate anonymised patient data across one or more healthcare networks for use in monitoring and/or improving healthcare services. Data indicative of the or each patient model may be maintained in a system internal or external to the healthcare network, and may be used for retrieving healthcare data for provisioning to user devices in one or more healthcare networks.

In examples, in response to receiving a request for healthcare data from the user device 101, the system 103 may identify at least one of the patient models in dependence on the requested healthcare data. For example, if the request relates to healthcare data related to a patient, then the system 103 may identify an individual patient model associated with the patient. The system 103 may retrieve the requested healthcare data using the identified patient model(s). The system 103 may identify the relevant of the known data sources using the known associations, and retrieve relevant healthcare data therefrom. The system 103 may transmit the retrieved healthcare data for receipt by the user device 101. Therefore, enabling retrieval of healthcare data based on patient models generated based on known associations.

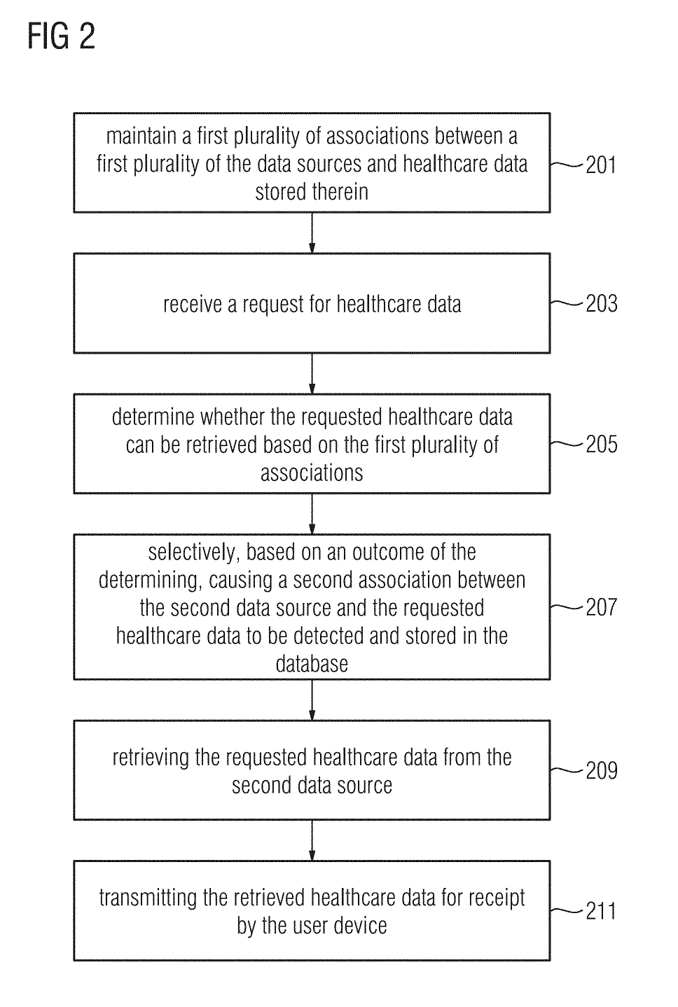

In the example set out with reference to FIG. 2, the system 103 provides healthcare data to the user device 101 or a support system (not shown), including, but not limited to, a population health management system, a clinical decision support system and/or a statistical decision support system. In this example, the system 103 maintains the known associations in the database 107 (block 201).

The system 103 may receive a request from the user device 101 or the support system (as appropriate) for healthcare data (block 203). The request may, for example, include data identifying a patient, a healthcare professional, a medical condition and/or another type of healthcare data for use by the system 103 in identifying relevant healthcare data. The request may, for example, be received by the system 103 in response to an event, including, but not limited to, a healthcare professional interacting with an application on the user device 101 or the support system (as appropriate).

In examples, the system 103 may be able to provide the requested healthcare data from the known data sources on the basis of the known associations. However, in examples, the requested healthcare data may be stored in a second of the data sources 107, wherein the second data source is different to the known data sources in the sense that it is either not known to the system 103 or mapping between the second data source and the requested healthcare data is not known to the system 103. In examples, the second data source is logically distinct to the first plurality of sources.

The system 103 may determine whether the requested healthcare data can be retrieved based on the known associations (block 205). The system 103 may, for example, identify relevant patient model(s) and use the identified patient models to determine whether the requested healthcare data can be retrieved. In the event that relevant patient model cannot be identified, the system 103 may cause a relevant patient model to be generated. In the event that the requested healthcare data can be retrieved on the basis of the known associations, the system 103 may retrieve the requested healthcare data and transmit it to the user device 101 or the support system (as appropriate).

In this example, the system 103 is unable to retrieve the requested healthcare data on the basis of the known associations. The system 103, based on an outcome of the determining step at block 205, causes a second association between the second data source and the requested healthcare data to be detected and stored in the database 107 (block 207), thereby causing the second association to be one of the known associations. As will be described in further detail below, association in relation to unknown or as yet undetected data sources may be detected using an application having, for example, crawling functionality.

In at least some examples, responsive to storing the detected association in respect of the second data source and the healthcare data stored therein, the system 103 may update one or more of the relevant patient models to include healthcare data contained in the newly detected second data source, thereby causing the patient model(s) to be automatically updated and be comprehensive. In examples, the second data source may be a newly added data source in the healthcare network 100.

The system 103 may retrieve the requested healthcare data from the second data source (block 209), and transmit it for receipt by the user device 101 or the support system (as appropriate) (block 211). In examples, the retrieved healthcare data may be anonymised for confidentiality before transmission to the user device 101 or the support system (as appropriate).

Therefore, examples flexibly integrate disparate data sources accessible via the healthcare network 100. The integration is based on knowledge regarding associations between data sources and healthcare data stored therein. Using this knowledge, the system 103 generates and maintains a virtual map of data sources for use in retrieval of healthcare data. The map is updated as unknown or undetected data sources are detected or in response to identifying additional healthcare data stored in a known data source. Thus, examples enable aggregation of data from disparate data sources in a healthcare network without having to store healthcare data in a centralised repository.

In at least some examples, the system 103 may cause the second association to be detected if an association corresponding to the healthcare data requested at block 203 is not determined on the basis of the known associations at block 205. In this case, the system 103 determines that the requested healthcare data cannot be retrieved based on the known associations, and selectively causes the second association to be detected based on the outcome of the determining at block 205.

If an association corresponding to the requested healthcare data is determined on the basis of the known associations at block 205, the system 103 may retrieve the healthcare data requested at block 203 on the basis of the relevant one of the known associations and transmit it to the user device 101 or the support system (as appropriate). Therefore, the system 103 selectively causes an association to be identified if the requested healthcare data cannot be retrieved based on the known associations.

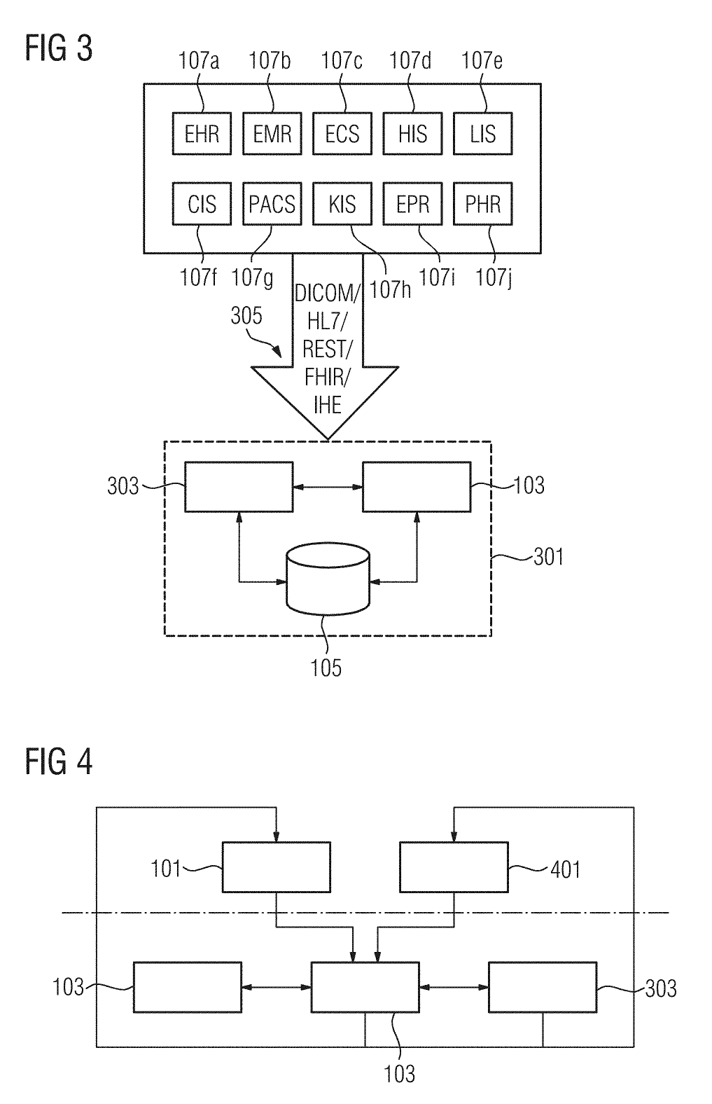

In the example set forth in FIG. 3, the system 103 uses an application 303 to detect which of the data sources 107 comprises the healthcare data requested at block 203, thereby causing detection of the second association. The application 303 may conceptually be a part of a healthcare data provisioning framework 301 configured to provision healthcare data to user devices and/or systems in one or more healthcare networks. The framework 301 additionally comprises the system 103 and the database 105.

In this example, the healthcare network 100 has access to: an EHR 107a, an EMR 107b, an ECS 107c, a HIS 107d, a LIS 107e, a CIS 107f, a PACS 107g, a KIS 107h, an EPR 107i and a PHR 107j. Communication between the data sources 107a-j may be via a standardised or a proprietary communications interface or an application programming interface 305. The interface may, for example, conform to one or more of standards: DICOM; HL7; REST; FHIR; IP; and/or IHE. In effect, the framework 301 communicates with the various data sources 107a-j using supported interfaces, but hides implementation specific details, including supported communication interfaces, from the user devices 101 and the support systems requesting healthcare data therefrom.

In examples, the application 303 may be a crawler (also referred to as bot or spider) comprising crawling functionality. Crawling process comprises detecting unknown data sources and/or new or updated content in known data sources. The application 303 may, additionally or alternatively, index healthcare data stored in newly detected data sources, thereby identifying relationship between a newly detected data source and healthcare data stored therein. The application may, additionally or alternatively, rank data sources based on, for example, reliability and healthcare data stored therein, and use the ranking data for filtering healthcare data when responding to requests for healthcare data. The application may, additionally or alternatively, use artificial intelligence and/or machine learning techniques to detect unknown data sources.

The application 303 may, additionally or alternatively, detect unknown data sources on the basis of a message queue, via which at least some of the data sources 107a-j announce or broadcast their presence. The application 303 may, additionally or alternatively, be configured to detect periodic beacon signals transmitted by one or more of the data sources 107a-j, in order to detect unknown data sources. For example, the application 303 may listen to an HL7 version 2 message queue and use the messages transmitted thereon them for notifying the system 303 of any updates in relation to known or unknown data sources and/or detecting unknown data sources.

The application 303 may, additionally or alternatively, be configured to conduct a capability negotiation exchange with at least some of the newly detected data sources 107a-j (as appropriate). The capability negotiation may, for example, involve detecting supported communication protocols, healthcare data stored and schema used for storing healthcare data, and may store it in a profile associated with the newly detected data source, thereby facilitating seamless access thereto. For example, as a consequence of the capability negotiation in respect of a newly discovered EMR data source, the application may identify that it is compatible with the FHIR and HL7 standards, and maintains records in respect of medical records in respect of patients admitted to the emergency unit in accordance with a schema specified in the FHIR standard.

In the example set forth in FIG. 4, the system 103 receives a request for healthcare data from the user device 101 or a decision support system 401. The system 103 may determine whether the requested healthcare data can be acquired on the basis of the known associations (i.e. the first plurality of associations and the second association). If the requested healthcare data can be provided on the basis of the known associations, then the system 103 retrieves the requested healthcare data on the basis of the known associations. If the requested healthcare data cannot be provided on the basis of the known associations, then the system 103 causes the application 303 to detect an association between one or more of the data sources 107 comprising the requested healthcare data and store it in the database 105, thereby causing the detected association to form part of the known associations.

In at least some examples, the system 103 may receive a further request for healthcare data from the same or different user device 101 or the support system 401 (as appropriate), wherein the requested healthcare data is stored in the second data source. In this case, the system 103 may retrieve the requested healthcare data on the basis of the known associations, which include the second association, and transmit it for receipt by the user device 101 or the decision support system 401 (as appropriate). Therefore, the system 103 adds the newly detected associations to known associations and use them for provisioning healthcare data.

In at least some examples, the system 103 may remove an association from the known associations, thereby ceasing access to all or part of healthcare data maintained in an associated data source via the healthcare network 100. The system may, for example, remove an association in response to a data source being replaced or being temporarily or permanently being taken out of service. Information regarding events that may cause an association to be permanently or temporarily removed may be acquired from the aforementioned message bus. The system 103 may, additionally or alternatively, in response to receiving data indicative of a change between the second data source and healthcare data stored therein, update the second association accordingly. Thus, the system 103 continues to facilitate access to the healthcare data stored in the second data source via the healthcare network 100.

In at least some examples, the system 103 creates one or more virtual maps based on the known associations indicative of links to the known data sources (i.e. the first plurality of data sources and the second data source) and healthcare data contained therein, thereby creating one or more patient models. As described above, a patient model may be an individual patient model, a collective patient model, a population patient model, a statistical patient model and/or a clinical patient model. In examples, the or each patient model may cause requests for healthcare data to be processed quicker because patient models map relevant healthcare data stored in disparate data sources.

In examples, one or more of the patient models may be a schema with relevant events, use cases and/or clinical processes represented as a dependency tree. In the example set forth in FIG. 5, an individual patient model 500 is represented as a dependency tree. In this example, a patient visits a healthcare professional with one or more symptoms (block 501). The healthcare professional inputs relevant notes via, for example, a user interface provided by an application on the user device 101 to be stored in one or more of the data sources 107 (block 502).

The healthcare professional may examine the patient (block 503) and may set up a diagnostic procedure involving, for example, imaging scans (such as a CT scan) and/or clinical tests (such as a blood test) (block 504). The diagnostic procedures may then be carried (blocks 506 and 507), and results may be made available to the same or another healthcare professional (block 505). Depending on the results and various assessments by healthcare professional(s), a medical condition may be identified (block 508) and a treatment plan may be created (block 509). The treatment plan in case of cancer may, for example, include surgery, chemotherapy and medication (blocks 510 and 511). The treatment plan may be revised based on how the patient is responding thereto. All the relevant information and dependencies are updated so that the patient model 500 is up-to-date.

Subsequent to conclusion of treatment, the patient may visit the same or another healthcare professional with the same or different set of symptoms (block 512) and notes regarding the visit may be entered for storage on the same or different of the data sources 107 (block 513). The healthcare professional may consult notes regarding the patient's history using the patient model 500, and may use that in addition to current symptoms for preparing a new diagnostic plan (block 515), which may then be executed at block 516 and relevant results updated at block 517. Based on the results of the diagnostic tests, an unrelated, a related or a comorbidity medical condition may be identified at block 514.

In at least some examples, the system 103 uses one or more of the patient models to identify relevant healthcare data at every stage of analysis for provisioning to a healthcare professional. This being the case, in the example set forth with reference to FIG. 5, the system 103 may control the display of the user device 101 to present different healthcare data to the healthcare professional in respect of at least some of the blocks 501 to 517. For example, a healthcare professional at block 512 may be presented with medical history concerning the patient for use in identifying any comorbidities, but a healthcare professional carrying out a diagnostic procedure at block 516 may not be presented with that information as it may not be relevant for that stage. Therefore, examples retrieve relevant healthcare data for a current stage of analysis from disparate data sources and present it, and thereby reduce the cognitive burden.

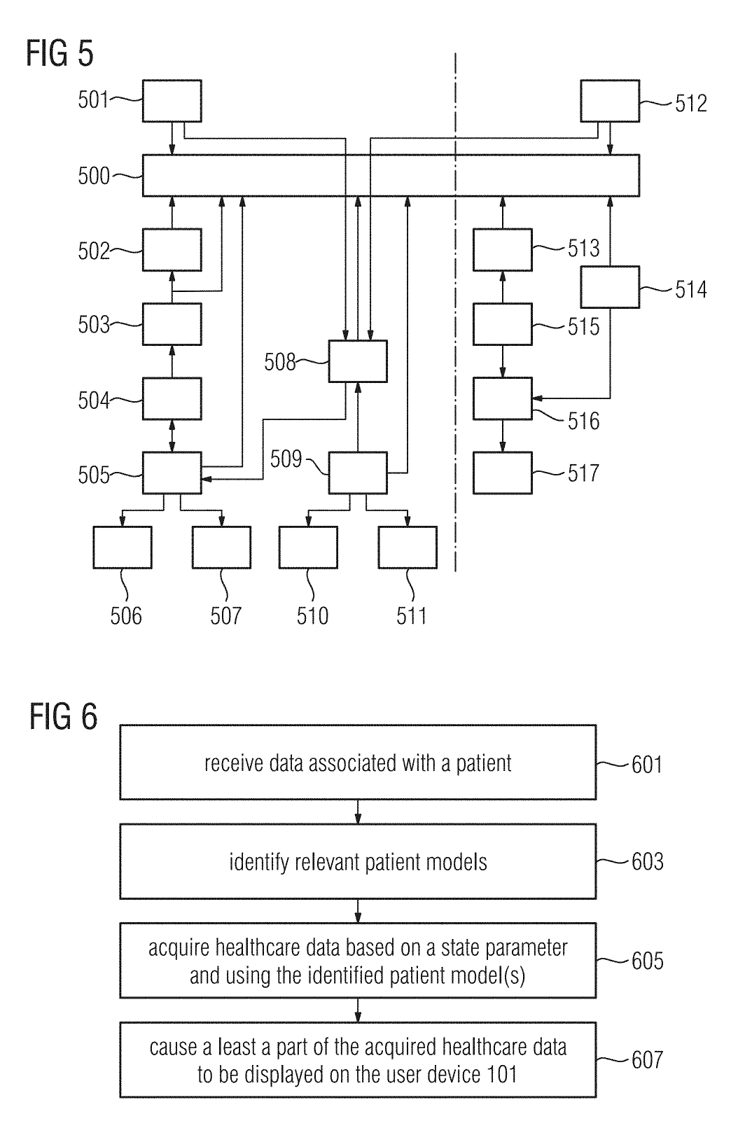

In the example set forth in FIG. 6, the system 103 may receive data, for example, associated with a patient and/or, a group of patients from the user device 101 or the support system 401 (block 601). The data may, for example, comprise data identifying the patient(s), symptoms, medical conditions and/or medical history. The system 103 may identify one or more of the relevant patient models (block 603), and cause the user device 101 to acquire relevant healthcare data based on a state parameter and using the identified patient model(s) (block 605). The state parameter may be indicative of one of different stages associated with analysis of the received healthcare data. For example, in the example set forth in FIG. 5 the state parameter may be indicative of one of the blocks 501-517. The system 103 may cause at least a part of the acquired healthcare data to be displayed on the user device 101 (block 607). The healthcare data may, for example, be selectively displayed based on an identity of a healthcare professional, and may be anonymised prior to presentation for confidentiality.

For example, a patient may identify an unusual lump in her breast and may visit a healthcare professional, such as a general practitioner, to get it checked. The system 103 may identify an individual patient model for the patient for use in retrieving relevant healthcare data to the healthcare professional. The healthcare professional may prepare a diagnostic plan for the patient, which may include mammogram, ultrasound, biopsy, scans and/or x-rays, to be carried out by the same or different healthcare professional(s). In addition, various medical records concerning the patient may be updated to identify potential ailments, such as breast cancer. Responsive to the updates to the various medical records, one or more related patient models may be updated. For example, data indicative of the patient or their medical records may be included in a patient model for patients suspected to have breast cancer.

The system 103 may, based on the diagnostic plan, cause follow-up appointments for the patient to be scheduled. The patient may visit healthcare professional(s) for diagnostic tests, and each of the healthcare professionals may presented with different relevant healthcare data. For example, the healthcare professional conducting mammogram may be presented with information such as whether the patient has had breast implants, but this information may not be presented to a healthcare profession carrying out a blood test. Therefore, relevant healthcare data concerning the patient is presented at every stage of assessment.

Following conclusion of the diagnostic plan, it may be confirmed that the patient indeed has breast cancer. The system 103 may update the relevant patient models following conclusion of the diagnostic test. The patient may be put on a therapy plan following conclusion of the diagnostic plan, which may, for example, involve radio, chemo, hormone and/or biological therapies. The healthcare professional(s) preparing the therapy plan may be presented with relevant healthcare data. The relevant data may include available clinical resources, guidelines and regulations retrieved using, for example, a population patient model. The relevant data may, additionally or alternatively, include data regarding success rates in various similar cases retrieved using, for example, a statistical and/or a clinical patient model. The relevant data may, additionally or alternatively, include results from various diagnostic tests retrieved using, for example, an individual patient model. The system 103 may cause the relevant patient models to be updated to include data indicative of the therapy plan and/or the patient's response thereto. Following the therapy, a care plan, including counselling and/or psychological care, may be devised for the patient.

In another example, a patient with persistent cough and/or breathlessness may be prescribed a diagnostic plan for suspected lung cancer patients. In this case, the system 103 may cause relevant healthcare data, such as risk factors, including history of smoking, may be presented to the healthcare professional. The diagnostic plan may be prepared to confirm existence of lung cancer and/or its stage, and may, for example, include x-ray, scans, bronchoscopy and/or biopsy. As described above, different healthcare data may be presented to healthcare professionals at different stages. Based on the results, a therapy plan may be devised for the patient, which may, for example, include surgery, radiotherapy and/or chemotherapy.

In another example, a patient with problems relating to urinating may be prescribed a diagnostic plan for suspected prostate cancer patients. In this case, the system 103 may cause relevant healthcare data, such as risk factors, including age, ethnicity, family history, weight and diet, may be presented to the healthcare professional. The diagnostic plan may be prepared to confirm existence of prostate cancer and/or its stage, and may, for example, include PSA testing, digital rectal examination and/or biopsy. As described above, different healthcare data may be presented to healthcare professionals at different stages. Based on the results, a therapy plan may be devised for the patient, which may, for example, include surgery, radiotherapy and/or chemotherapy.

In some arrangements, the system 103 can monitor prior actions of one or more healthcare professionals when analysing healthcare data associated with at least some of the different stages. The prior actions may be monitored when, for example, the healthcare professional is analysing healthcare data in respect of the or another patient. The system 103, based on the monitoring of the prior actions and the state parameter, causes at least a part of the healthcare data acquired at block 605 to be displayed on the user device 101 in a tailored manner. Therefore, examples customise the information presented in accordance with healthcare professionals' style of working, and as a consequence are capable of personalising display of healthcare data.

The system 103 may, additionally or alternatively pro-cess the healthcare data acquired at block 605 based on the monitoring of the prior actions. For example, if the system 103 determines that a healthcare professional changes the scanned images to a certain resolution, then the system 103 may process subsequently received scanned images to that same resolution when presenting them to the healthcare professional. The system 103 may then cause at least part of processed healthcare data to be displayed on the user device 101.

In at least some examples, the system 103, in response to receiving a user input, for example, from a healthcare professional via the user device 101 or the support system 401, may cause the user device 101 or the support system 401 to acquire further healthcare data based on a combination of the received user input and the state parameter, and using the patient model(s) identified at block 603. The system 103 may cause the user device 101 to present at least a part of the further healthcare data. Therefore, examples update relevant healthcare data based on the healthcare professional's interaction in connection with the healthcare data that is currently presented.

In at least some examples, the system 103 may cause the user device 101 to process at least a part of the healthcare data acquired at block 605 based on a predetermined parameter, and thereafter causes at least a part of the processed healthcare data to be displayed on the user device 101. The predetermined parameter may, for example, comprise or be based on: data associated with the patient usable for processing, for example, allergies; data associated with another patient usable for processing, for example, similarities and/or comorbidities; data indicative of a healthcare user or professional usable for processing healthcare data in accordance with, for example, personal preferences; data indicative of a medical condition usable for processing healthcare data related to, for example, statistical or clinical decision support; data indicative of one or more guidelines usable for processing healthcare data to comply with, for example, relevant guidelines; data indicative of one or more legal regulations usable for processing healthcare data in accordance with, for example, relevant legislations; data indicative of a customisation parameter usable for processing healthcare data in accordance with, for example, local customisations; and/or data indicative of a user setting usable for processing healthcare data in accordance with, for example, user settings. Therefore, examples pre-process acquired healthcare data and thereby reduce the burden.

In at least some examples, the system 103 may maintain a context parameter indicative of a clinical step associated with the healthcare data received at block 601. For example, the context parameter in case of a patient undergoing bronchoscopy may be indicative of a current clinical step in the procedure: premedication, sedation, administration of oxygen, brushing, biopsy, etc. The system 103 may filter the healthcare data acquired at block 605 based on the context parameter, and cause at least a part of the filtered healthcare data to be displayed on the user device 101 or the support system 401. In examples, the healthcare data may, additionally or alternatively, be filtered based on: a medical condition or disease; the patient being examined; a healthcare profession assessing the information presented; a current stage of assessment; and/or a clinical path. Filtering may be performed in stages, such as filtering based on medical condition followed by data specific to the patient being examined. Therefore, examples present relevant healthcare data for a current step in a clinical process by filtering out irrelevant information.

The system 103 may control display of at least part of the filtered healthcare data to be displayed on the user device 101 or the support system 401 based on a combination of the context parameter and the state parameter. For example, the state parameter may be indicative of a clinical process, such as treatment stage bronchoscopy, and the context parameter may be indicative of the current step in the procedure, such as premedication. The system 103 may, additionally or alternatively, customise the information presented in accordance with an associated frame of reference, such as a healthcare professional associated with a current stage. For example, a comorbidity for a lung cancer patient can be diabetes, so it is important that this information is presented to a healthcare profession generating a treatment or diagnosis plan but not as important for a healthcare professional carrying out a diagnostic test in accordance with the diagnostic plan.

In examples, the system 103 may link the healthcare data acquired at block 605, and present a pictorial or graphical representation thereof, thereby reducing the burden. The representation may, for example, be in the form of a timeline, a clinical path, a decision tree and/or a dependency tree.

In some arrangements, the healthcare data is acquired at block 605 based on: data indicative of a patient; data indicative of a healthcare user or professional; data indicative of a medical condition; data indicative of one or more guidelines; data indicative of one or more legal regulations; data indicative of a customisation parameter; and/or data indicative of a user setting. Therefore, examples acquire relevant healthcare data based on associated frame of reference and/or user preference.

In examples, the healthcare data acquired at block 605 comprises data indicative of: healthcare data associated with a patient being examined; healthcare data associated with one or more further patients; and/or expert medical knowledge. The system 103 may, for example, cause further relevant healthcare data related to the patient to be acquired to aid analysis and/or for analysis in relation to a comorbidity. The system 103 may, additionally or alternatively, retrieve healthcare data corresponding to other patients with similar symptoms and/or conditions. The system 103 may, additionally or alternatively, retrieve expert healthcare data from, for example, scientific journals to aid a healthcare professional. Therefore, examples provide comprehensive healthcare data at every stage.

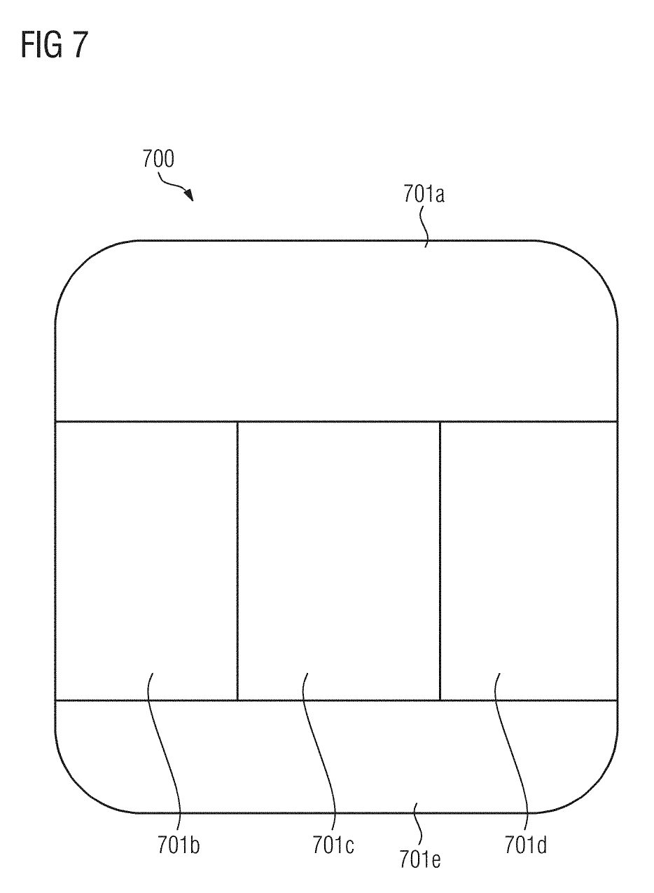

In the example set forth in FIG. 7, an interface 700 provided by an application on the user device 101 for presentation of healthcare data acquired at block 605 or otherwise acquired. The healthcare data may correspond to one or more patients, and may be presented for use in making clinical, administrative and/or management decisions.

In this example, the interface 700 is split into zones 701a-e for presenting different types of healthcare data. Healthcare data may be organised in the zones based on user customisations and/or system settings. For example, the system 103 may cause a timeline indicative of a patient's medical history or a clinical path to be generated based on the healthcare data acquired at block 605 and presented in the zone 701a to present an overview their medical history to a healthcare professional.

In the zone 701b the system 103 may cause the interface 700 to present symptoms being experienced and other metadata, such as age, risks and allergies, related to the patient.

In the zone 701c the system 103 may cause the interface 700 to present a pictorial representation of the affected organs and regions thereof, and/or images from scans conducted during the diagnostic stage. For example, in respect of a patient with brain tumour the system 103 may cause an image of a brain identifying location of one or more tumours to be displayed in the zone 701c. The system 103 may, additionally or alternatively, cause a relevant dependency or decision tree to be displayed in the zone 701c to aid a healthcare professional with his assessment

In the zone 701d the system 103 may cause the interface 700 to present relevant information from a statistical and/or a clinical decision support system.

In the zone 701e the system 103 may cause the interface 700 to present relevant healthcare data based on the aforementioned context parameter, such as data identifying a healthcare professional and/or a current stage of analysis.

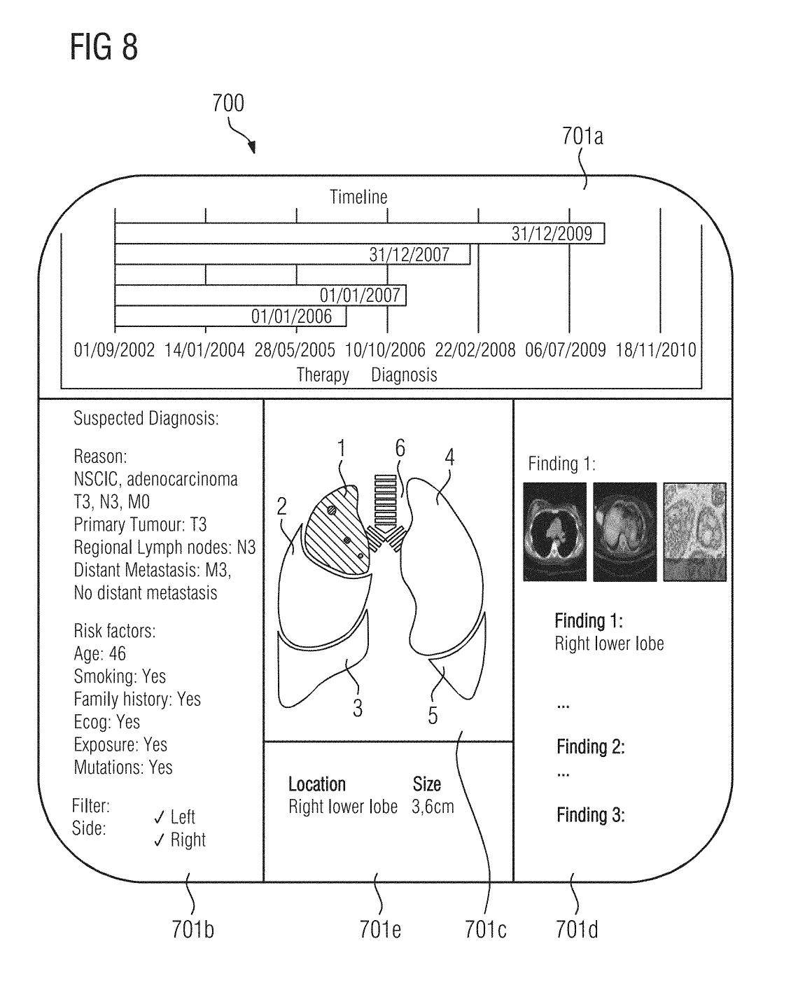

In the example set forth in FIG. 8, the user interface 700 is customised for a healthcare professional devising a therapy plan for a patient with lung cancer in the right lower lobe. In this example, the healthcare professional is presented with a timeline in the zone 701a setting out time periods associated with the condition and the treatment process. In examples, the zone 701a may, additionally or alternatively, include links to other parts of the interface 700 configured to present related healthcare information, such as status of the treatment plan, diagnostic procedures and their results, and details regarding the therapy plan. In examples, the zone 701a may, additionally or alternatively, include a link to the healthcare professional's other patients, appointments diary and/or relevant guidelines and regulations.

In the zone 701b, relevant healthcare data, such as details of the diagnosis and risk factors may be presented. In addition, may include means for filtering the presented information, such as check boxes that enable the healthcare professional to limit the information displayed, such as limiting healthcare data to right and/or left side of the lung.

In the zone 701c, a pictorial representation of a lung identifying location of the cancer may be displayed. In the zone 701d, data indicative of the various diagnostic tests and/or their findings may be displayed. In the zone 701e relevant metadata regarding the cancer, such as type, size and location may be displayed.

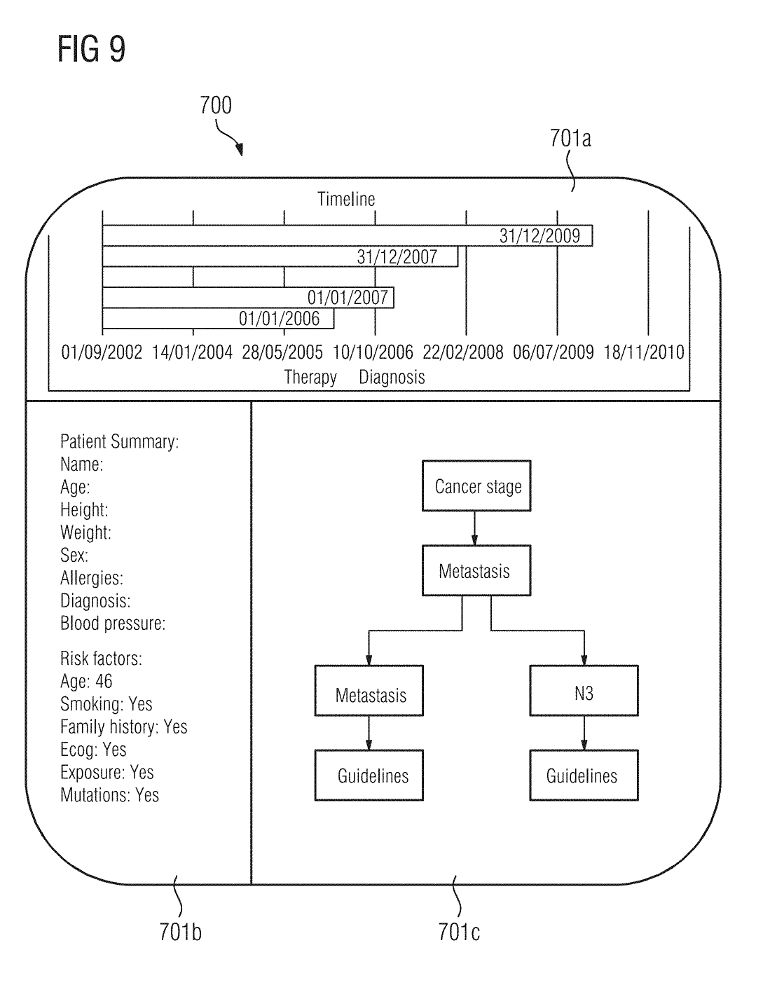

In the example set forth in FIG. 9, the user interface 700 is customised to display status information regarding a case involving a patient with lung cancer. In this example, the timeline indicative of the clinical process is displayed in the zone 701a. In the zone 701b, relevant metadata related to the patient is displayed along with the risk factors. In the zone 701c a workflow representing various clinical stages is displayed along with links to guidelines relevant for a current stage of examination.

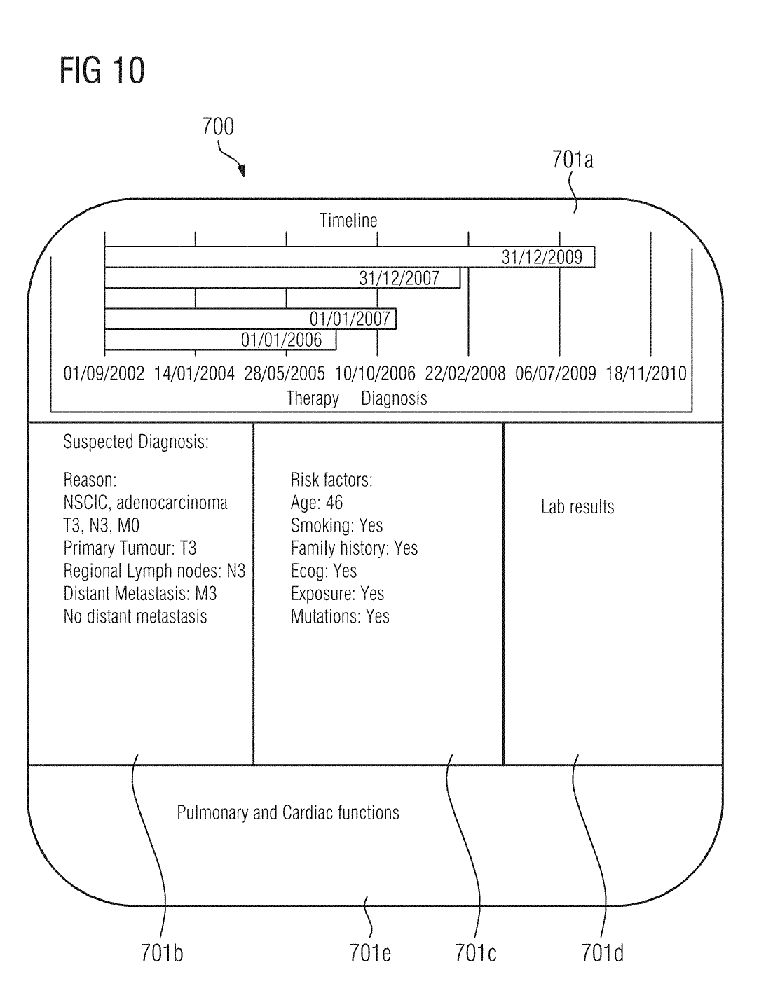

In the example set forth in FIG. 10, the user interface 700 is customised to display status information regarding a case involving a patient with lung cancer. In this example, the timeline indicative of the clinical process is displayed in the zone 701a. In the zone 701b, relevant metadata related to the patient is displayed. In the zone 701c risk factors associated with the patient are displayed. In the zone 701d, data indicative of results from the various lab and diagnostic tests are displayed. In the zone 701e data indicative of the status of the therapies, such as concomitant cardiac and pulmonary operations, are displayed.

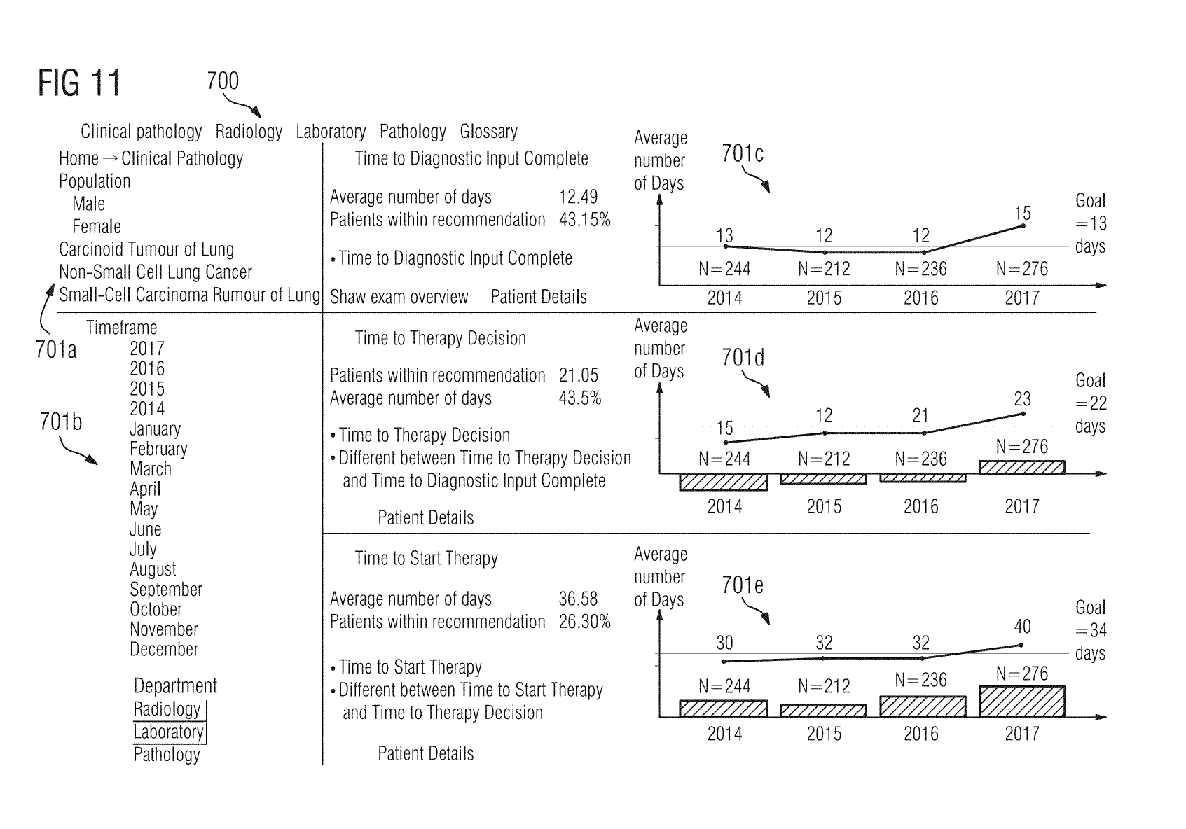

In the example set forth in FIG. 11, the user interface 700 is customised to display data indicative of population health management for use in making administrative and/or managerial decisions, and/or monitoring performance. In this example, various metrics relating to lung cancer patients are displayed. The user interface 700 includes means for controlling information displayed in the various zones 701, such as links, at the top, and these include limiting information relevant to “Clinical Pathology”, “Radiology”, “Laboratory”, and “Pathology”. The links may additionally include pointers to reference information, such as a link to “Glossary”.

In this example, information relevant to “Clinical Pathology” is displayed in the various zones 701. In the zone 701a metadata relating to a proportion of patient population selected for use in preparing the various displayed metrics. The zone 701b includes means for restricting the proportion of patient population selected for use in preparing the various displayed metrics, for example, by restricting the timeframe for assessment. For example, the healthcare professional may restrict the displayed metrics patients being treated in the year 2017.

The user interface 700 may present metadata and a graph relating to each of the metrics selected for display in the zones 701c-e. In this example, the zone 701c displays metadata and a graph relating to average time taken for the clinical stage “TIME TO DIAGNOSTIC INPUT COMPLETE”, the zone 701d displays metadata and a graph relating to average time taken for the clinical stage “TIME TO THERAPY DECISION”, and the zone 701e displays metadata and a graph relating to average time taken for the clinical stage “TIME TO START OF THERAPY”. Management of the healthcare network 100 may, for example, use this information for assessing whether key targets have been met. Therefore, the user interface 700 is customised based on healthcare professionals' role and task.

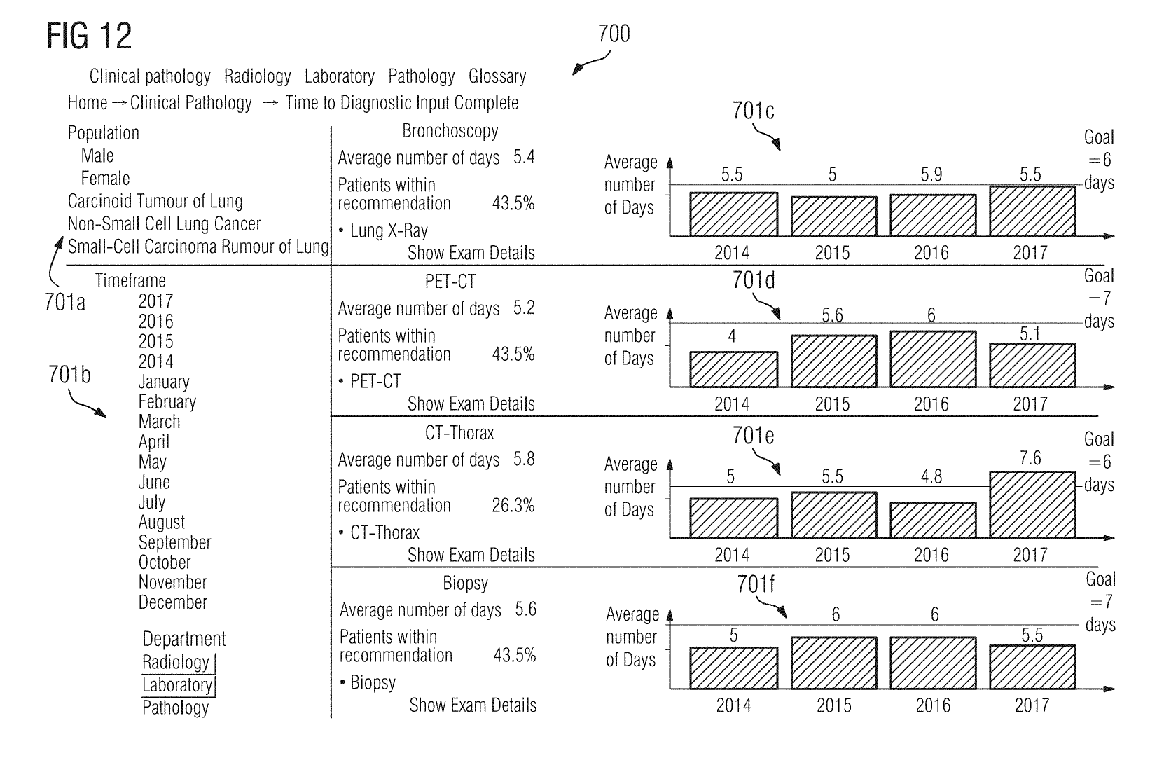

In the example set forth in FIG. 12, the user interface 700 is customised to display data indicative of population health management for use in making administrative and/or managerial decisions, and/or monitoring performance. In this example, various metrics relating to lung cancer patients are displayed. Similar to the example set forth in FIG. 12, the user interface 700 includes means for controlling information displayed in the various zones 701, such as links, at the top, and these include limiting information relevant to “Clinical Pathology”, “Radiology”, “Laboratory”, and “Pathology”. The links may additionally include pointers to reference information, such as a link to “Glossary”.

In this example, information relevant to “Clinical Pathology” is displayed in the various zones 701, specifically information relating to the various diagnostic procedures in respect of lung cancer. In examples, the healthcare professional may navigate to the display in accordance with FIG. 12 from one or more links presented in the example set forth in FIG. 11, such as the link “Laboratory”.

In this example, in the zone 701a metadata relating to a proportion of patient population selected for use in preparing the various displayed metrics. The zone 701b includes means for restricting the proportion of patient population selected for use in preparing the various displayed metrics, for example, by restricting the timeframe for assessment. For example, the healthcare professional may restrict the displayed metrics patients being treated in the year 2017.

The user interface 700 may present metadata and a graph relating to each of the metrics selected for display in the zones 701c-f. In this example, the zone 701c displays metadata and a graph relating to average time taken for the diagnostic procedure “Bronchoscopy”, the zone 701d displays metadata and a graph relating to average time taken for the diagnostic procedure “PET-CT”, the zone 701e displays metadata and a graph relating to average time taken for the diagnostic procedure “CT-Thorax”, and the zone 701f displays metadata and a graph relating to average time taken for the diagnostic procedure “Biopsy”. Management of the healthcare network 100 may, for example, use this information for resource management.

Therefore, the framework enables a homogeneous environment in which links to disparate data sources are maintained and updated based on information required, thereby integrating disparate data sources. The retrieved information is based on relevant context, and is automatically updated as the analysis advances. In addition, presented information is customised in accordance with preferences of healthcare professionals′, thereby improving user experience.

The above are to be understood as illustrative examples. Further examples are envisaged.

In examples described above, the healthcare network 100 comprises one or more user devices 101, a system 103, a database 105 and one or more data sources 107. In other examples, the healthcare network comprises additional systems.

It is to be understood that any feature described in relation to any one example may be used alone, or in combination with other features described, and may also be used in combination with one or more features of any other of the examples, or any combination of any other of the examples. Furthermore, equivalents and modifications not described above may also be employed without departing from the scope of the invention, which is defined in the accompanying claims.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. § 112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A system operable to provide healthcare data to a user device in a healthcare network, the healthcare network including a plurality of data sources including healthcare data, the system comprising:
at least one processor; and
at least one memory storing computer program code, the at least one memory and the computer program code being configured to, with the at least one processor, cause the system to at least:
maintain, in a database, a first plurality of associations between a first plurality of the data sources and healthcare data stored in the database;
receive, from the user device, a request for healthcare data, wherein the healthcare data requested is stored in a second data source, the second data source being different from the first plurality of data sources;
determine whether the healthcare data requested is retrievable based on the first plurality of associations;
selectively, based on a result determined, causing a second association between the second data source and the healthcare data requested to be detected and stored in the database;
retrieve the healthcare data requested from the second data source; and
transmit the healthcare data retrieved for receipt by the user device.

2. The system of claim 1, wherein the at least one memory and the computer program code are configured to, with the at least one processor, cause the system to:
cause the second association to be detected, in response to an association corresponding to the healthcare data requested not being determined based upon the first plurality of associations.

3. The system of claim 1, wherein the at least one memory and the computer program code are configured to, with the at least one processor, cause the system to:
retrieve, in response to an association corresponding to the healthcare data requested being determined based upon the first plurality of associations, the healthcare data requested based upon the association determined; and
transmit the healthcare data retrieved for receipt by the user device.

4. The system of claim 1, wherein the at least one memory and the computer program code are configured to, with the at least one processor, cause the system to:
use an application to detect at least one of the data sources including the healthcare data requested, thereby detecting the second association.

5. The system of claim 4, wherein the application includes crawling functionality.

6. The system of claim 1, wherein the at least one memory and the computer program code are configured to, with the at least one processor, cause the system to:
receive, from the user device, a request for further healthcare data, wherein the further healthcare data requested is stored in the second data source;
retrieve the further healthcare data requested based on the second association; and
transmit the further healthcare data retrieved for receipt by the user device.

7. The system of claim 1, wherein the at least one memory and the computer program code are configured to, with the at least one processor, cause the system to:
remove an association from the first plurality of associations, thereby ceasing access to the corresponding healthcare data via the healthcare network.

8. The system of claim 1, wherein the at least one memory and the computer program code are configured to, with the at least one processor, cause the system to:
update the second association, in response to receiving data indicative of a change between the second data source and healthcare data stored in the second data source, thereby continuing to facilitate access to the healthcare data stored in the second data source via the healthcare network.

9. The system of claim 1, wherein the at least one memory and the computer program code are configured to, with the at least one processor, cause the system to:
create a virtual map of the first plurality of data sources and the second data source based on the first plurality of associations and the second association, thereby creating a patient model.

10. The system of claim 9, wherein the at least one memory and the computer program code are configured to, with the at least one processor, cause the system to:
cause the user device to acquire, in response to receiving data associated with a patient, healthcare data based on a state parameter and using the patient model, the state parameter being indicative of one of different stages associated with analysis of the healthcare data acquired; and
cause at least a part of the healthcare data acquired to be displayed on the user device.

11. The system of claim 10, wherein the at least one memory and the computer program code are configured to, with the at least one processor, cause the system to:
monitor prior actions of a user when analysing healthcare data associated with at least some of the different stages; and
control display of at least a part of the healthcare data acquired on the user device based on a combination of the state parameter and the prior actioned monitored.

12. The system of claim 10, wherein the at least one memory and the computer program code are configured to, with the at least one processor, cause the system to:
cause the user device to acquire further healthcare data, in response to a user input, based on a combination of the user input and the state parameter, and using the patient model; and
cause the user device to present at least a part of the further healthcare data acquired.

13. The system of claim 10, wherein the at least one memory and the computer program code are configured to, with the at least one processor, cause the system to:
cause the user device to process the healthcare data acquired based on a parameter; and
cause at least a part of the healthcare data processed to be displayed on the user device.

14. The system of claim 13, wherein the parameter comprises at least one of:
data associated with the patient;
data associated with another patient;
data indicative of a healthcare user;
data indicative of a medical condition;
data indicative of one or more guidelines;
data indicative of one or more legal regulations;
data indicative of a customisation parameter; and
data indicative of a user setting.

15. The system of claim 10, wherein the at least one memory and the computer program code are configured to, with the at least one processor, cause the system to:
maintain a context parameter indicative of a clinical step associated with analysis of the healthcare data acquired;
filter the healthcare data based on the context parameter; and
cause at least a part of the healthcare data filtered to be displayed on the user device.

16. The system of claim 15, wherein the at least one memory and the computer program code are configured to, with the at least one processor, cause the system to perform:
control display of at least a part of the healthcare data filtered, on the user device, based on a combination of the state parameter and the context parameter.

17. The system of claim 10, wherein the healthcare data is acquired based on at least one of:
data indicative of the patient;
data indicative of a healthcare user;
data indicative of a medical condition;
data indicative of one or more guidelines;
data indicative of one or more legal regulations;
data indicative of a customisation parameter; and
data indicative of a user setting.

18. The system of claim 10, wherein the healthcare data acquired comprises data indicative of at least one of:
healthcare data associated with the patient;
healthcare data associated with another patient; and
expert medical knowledge.

19. The system of claim 1, wherein the data sources comprise at least one of:
an Electronic Medical Records data source;
a Picture Archiving Communication Systems data source;
an Emergency Care Summary data source;
a Key Information Summary data source;
an Electronic Healthcare Record data source;
an Electronic Patient Record data source; and
a Personal Health Record data source.

20. The system of claim 1, wherein the system is configured to communicate with a first of the data sources using an application programming interface in accordance with a Fast Healthcare Interoperability Resources standard.

21. A method of providing healthcare data to a user device in a healthcare network, the healthcare network including a plurality of data sources including healthcare data, the method comprising:
maintaining, in a database, a first plurality of associations between a first plurality of the data sources and healthcare data stored in the database;
receiving, from the user device, a request for healthcare data, wherein the healthcare data requested is stored in a second data source, different from the first plurality of data sources;
determining whether the healthcare data requested is retrievable based on the first plurality of associations;
selectively, based on a result of the determining, causing a second association between the second data source and the healthcare data requested to be detected and stored in the database;
retrieving the healthcare data requested from the second data source; and
transmitting the healthcare data retrieved for receipt by the user device.

22. A non-transitory computer readable medium storing a computer program, including a set of instructions which, when executed by a computerized device, cause the computerized device to perform a method of providing healthcare data to a user device in a healthcare network, the healthcare network comprising a plurality of data sources comprising healthcare data, the method comprising:
maintaining, in a database, a first plurality of associations between a first plurality of the data sources and healthcare data stored in the database;
receiving, from the user device, a request for healthcare data, wherein the healthcare data requested is stored in a second data source, different from the first plurality of data sources;
determining whether the healthcare data requested is retrievable based on the first plurality of associations;
selectively, based on a result of the determining, causing a second association between the second data source and the healthcare data requested to be detected and stored in the database;
retrieving the healthcare data requested from the second data source; and
transmitting the healthcare data retrieved for receipt by the user device.

23. The system of claim 2, wherein the at least one memory and the computer program code are configured to, with the at least one processor, cause the system to:
retrieve, in response to an association corresponding to the healthcare data requested being determined based upon the first plurality of associations, the healthcare data requested based upon the association determined; and
transmit the healthcare data retrieved for receipt by the user device.

24. The system of claim 2, wherein the at least one memory and the computer program code are configured to, with the at least one processor, cause the system to:
use an application to detect at least one of the data sources including the healthcare data requested, thereby detecting the second association.

25. The system of claim 24, wherein the application includes crawling functionality.

26. The system of claim 2, wherein the at least one memory and the computer program code are configured to, with the at least one processor, cause the system to:
receive, from the user device, a request for further healthcare data, wherein the further healthcare data requested is stored in the second data source;
retrieve the further healthcare data requested based on the second association; and
transmit the further healthcare data retrieved for receipt by the user device.

27. The system of claim 3, wherein the at least one memory and the computer program code are configured to, with the at least one processor, cause the system to:
receive, from the user device, a request for further healthcare data, wherein the further healthcare data requested is stored in the second data source;
retrieve the further healthcare data requested based on the second association; and
transmit the further healthcare data retrieved for receipt by the user device.

28. The system of claim 2, wherein the at least one memory and the computer program code are configured to, with the at least one processor, cause the system to:
update the second association, in response to receiving data indicative of a change between the second data source and healthcare data stored in the second data source, thereby continuing to facilitate access to the healthcare data stored in the second data source via the healthcare network.

29. The system of claim 11, wherein the at least one memory and the computer program code are configured to, with the at least one processor, cause the system to:
cause the user device to acquire further healthcare data, in response to a user input, based on a combination of the user input and the state parameter, and using the patient model; and
cause the user device to present at least a part of the further healthcare data acquired.

30. The system of claim 11, wherein the at least one memory and the computer program code are configured to, with the at least one processor, cause the system to:
cause the user device to process the healthcare data acquired based on a parameter; and
cause at least a part of the healthcare data processed to be displayed on the user device.

31. The method of claim 21, further comprising:
causing the second association to be detected, in response to an association corresponding to the healthcare data requested not being determined based upon the first plurality of associations.

32. The method of claim 21, further comprising:
retrieving, in response to an association corresponding to the healthcare data requested being determined based upon the first plurality of associations, the healthcare data requested based upon the association determined; and
transmitting the healthcare data retrieved for receipt by the user device.

33. The non-transitory computer readable medium of claim 22, wherein the method further comprises:
causing the second association to be detected, in response to an association corresponding to the healthcare data requested not being determined based upon the first plurality of associations.

34. The non-transitory computer readable medium of claim 22, wherein the method further comprises:
retrieving, in response to an association corresponding to the healthcare data requested being determined based upon the first plurality of associations, the healthcare data requested based upon the association determined; and
transmitting the healthcare data retrieved for receipt by the user device.
